(19) United States
(12) Patent Application Publication
(10) Pub. No.: US 2025/0049403 A1
(43) Pub. Date: Feb. 13, 2025

(54) METHOD FOR AUTOMATICALLY SETTING IMAGE ACQUISITION PARAMETERS, MEDICAL OBJECT, AND MEDICAL X-RAY SYSTEM

(71) Applicant: Siemens Healthineers AG, Forchheim (DE)

(72) Inventor: Marcus Pfister, Bubenreuth (DE)

(21) Appl. No.: 18/785,304

(22) Filed: Jul. 26, 2024

(30) Foreign Application Priority Data
Aug. 9, 2023 (DE) 10 2023 207 635.7

(51) Int. Cl. A61B 6/12; A61B 6/00

(52) U.S. Cl. CPC A61B 6/12; A61B 6/485

(57) ABSTRACT

A method is provided for automatically setting image acquisition parameters of a medical X-ray system when acquiring X-ray images of a patient with an object arranged on or in the body of the patient. The method includes: providing an X-ray image of the object, wherein the object has X-ray visible markings on its surface with encoded information, in particular material data, relating to the object; determining the X-ray visible markings of the X-ray image of the object, (e.g., by image recognition); evaluating the X-ray visible markings with regard to the information contained therein relating to the object, (e.g., material data); assigning image acquisition parameters to the information; and automatically setting the assigned image acquisition parameters on the X-ray system.

The present patent document claims the benefit of German Patent Application No. 10 2023 207 635.7, filed Aug. 9, 2023, which is hereby incorporated by reference in its entirety.

### TECHNICAL FIELD

The disclosure relates to a method for automatically setting image acquisition parameters when acquiring X-ray images of a patient with an object arranged on or in the body of the patient, a medical object, and a medical X-ray system for carrying out such a method.

### BACKGROUND

In known minimally invasive, X-ray-guided procedures, guide wires and catheters are inserted into the blood vessels via small openings in the vascular system (for example, in the groin), by which therapies may then be carried out, (e.g., the insertion of angioplasty). C-arm angiography systems may be used for imaging. Advancing technologies, surgical techniques, and the further development of instruments enable ever increasing complex procedures to be carried out in this way. The procedures are also becoming longer and longer, which means that more radiation is applied. The leads to an increased radiation loading on patients and personnel.

For example, through the development of special settings and optimizations of parameters such as for example filtering, etc., through to the adjustment of X-ray parameters to specific materials to be imaged on the X-ray image, X-ray imaging continues to develop in the direction of “low dose,” e.g., the lowest possible X-ray dose with the highest possible image quality. However, increasingly more complex procedures and (manual) system operation may easily lead to the selection of less than optimal fluoroscopy parameters. It is known to select the X-ray imaging parameters manually or automatically depending on the examination method (for example, fluoroscopy or 3D imaging), for example, via a menu in the imaging program.

It is known to apply markings to instruments or objects to make them easier to identify on an X-ray image. Standardized position markers on fenestrated aortic stents, for example, are prior art. Moreover, it is known to provide binary information encoding, for example, of patient data on medical objects to be implanted, such as stents (see, e.g., M. Pfister, “Binäre Informationskodierung auf Stents oder Devices, z.B. für Aortenstenting,” 2014, [Binary information encoding on stents or devices, for example for aortic stenting, 2014] https://portal.dnb.de/opac.htm?method=simpleSearch&cqlMode=true&query=idn%3D1064138721).

### SUMMARY AND DESCRIPTION

The object of the present disclosure is to provide a method, which, during interventional procedures using medical objects that are introduced into the body of a patient, enables the object to be recognized as well as possible and the radiation exposure to be as low as possible under X-ray imaging. Furthermore, it is the object of the disclosure to provide a medical system suitable for carrying out the method.

The object is achieved by a method for automatically setting image acquisition parameters when acquiring X-ray images of a patient with an object arranged on or in the body of the patient, by a medical object, and by a medical X-ray system as disclosed herein.

The scope of the present disclosure is defined solely by the appended claims and is not affected to any degree by the statements within this summary. The present embodiments may obviate one or more of the drawbacks or limitations in the related art.

The method for automatically setting image acquisition parameters of a medical X-ray system when acquiring X-ray images of a patient with at least one object arranged on or in the body of the patient, includes: a) providing an X-ray image of the object, wherein the object has X-ray visible markings on its surface with encoded information, (e.g., material data), relating to the object; b) determining the X-ray visible markings of the X-ray image of the object, (e.g., by image recognition); c) evaluating the X-ray visible markings with regard to the information contained therein relating to the object; d) assigning image acquisition parameters to the information; and e) automatically setting the assigned image acquisition parameters on the X-ray system.

In particular, the encoded information contains material data, (e.g., data relating to the materials and compounds contained or their exact composition). Other intervention-relevant information may also be included. For example, information relating to an advantageous image or representation of the implant may be present, such as a particularly advantageous angulation or projection direction.

This makes it possible during interventional procedures both to optimize image quality and minimize the X-ray dose quickly and easily and without any additional work step for the operator during X-ray imaging. The system automatically sets the optimum X-ray parameters for the medical object to be inserted or its material composition in order to make the object particularly easy to localize and recognize on an X-ray image. Complex manual readjustments are no longer necessary, and the X-ray dose may be kept low, which is beneficial for the health of the patient and the operator.

According to one embodiment, at least one further X-ray image is subsequently acquired using the image acquisition parameters set in this way. This may be realized, for example, if the method is carried out during or at the beginning of a fluoroscopy sequence (e.g., sequence of 2D fluoroscopic images). However, it may also be used for 3D X-ray imaging.

According to a further embodiment, the information is encoded as a one-dimensional bar code, multi-dimensional bar code, binary code, or another machine-readable code. A bar code is an opto-electronically readable script, which may include parallel black bars and spaces of different widths (one-dimensional). Alternatively, the bar codes may be colored, two-dimensional, or have a different pattern. Bar codes are well-known, easy to apply optically, and may also be read quickly and easily.

According to a further embodiment, the assignment is carried out based on a comparison with a stored list or with data from a database. Such lists with X-ray parameters optimized for the corresponding material may be stored locally, (e.g., in a memory of the X-ray system), or in an external, also publicly accessible database. The lists may be created beforehand from empirical values or simulations or may be retrieved from known lists and optimized. The lists may advantageously contain further selection parameters, for example, the type of X-ray system, the type of exposure (for example, whether 2D or 3D X-ray imaging is used) or information relating to the patient (for example, patient size, age, weight, etc.). These may also be selected automatically based on additional information that is stored or obtained via sensors, for example, or user input may be requested in this respect.

According to a further embodiment, the image acquisition parameter adapted to the material of the object may include an X-ray current, an X-ray voltage, an X-ray dose, an exposure time, a filter setting, a collimator setting, an X-ray pulse length, or a combination thereof. Multiple or all of these X-ray parameters may also be used.

According to a further embodiment, the at least one further acquired X-ray image may be used for a repetition of acts b) to e). This may be used quickly and easily, in particular for a series of fluoroscopy images (radioscopic images), in order to avoid errors during readout and, for example, to optimize for the correct object to be imaged in the case of multiple objects. The method may also be carried out multiple times in succession in this way. In particular, the method may be repeated iteratively until a cancellation criterion is achieved. Such a cancellation criterion may be a user input or also the end of a fluoroscopy sequence or of the procedure.

The disclosure further includes an object for insertion into the body of a patient. The object includes at least one X-ray visible marking, wherein the marking includes encoded information, (e.g., material data), relating to the object. Such an object may be a catheter, a guide wire, an implantable object such as a stent or a MitraClip, or a surgical instrument such as an ablation tool. The marking is X-ray visible, i.e., the marking may be recognized on an X-ray image, for example, via image recognition or another optical readout option. It may be a bar code, 2D matrix bar code, other binary code, or other machine-readable code, for example. The marking may be arranged so that it is recognizable from all sides or may be arranged multiple times on different sides. The information relates in particular to the material data, such as the materials, compounds, and exact compositions contained. Other intervention-relevant information may also be included. For example, information relating to an advantageous image or representation of the implant may be present, such as a particularly advantageous angulation or projection direction.

The disclosure also includes a medical system for carrying out the method using an object described above. The medical system includes: an X-ray system for acquiring X-ray images of the object; a system control unit for setting X-ray parameters for the X-ray system; an image system with an algorithm for carrying out image recognition, an evaluation unit for evaluating the X-ray visible markings with regard to information relating, in particular, to material data of the object; an assignment unit for assigning image acquisition parameters to the material data; and a memory having a stored list or data link to a database with image acquisition parameters assigned to the material data for the X-ray system.

### BRIEF DESCRIPTION OF THE DRAWINGS

The disclosure and further advantageous embodiments are explained in detail below with the aid of schematically illustrated embodiments in the drawings, without the disclosure thereby being limited to these embodiments. In the drawings:

FIG. 1 depicts an example of acts of the method for automatically setting image acquisition parameters of a medical X-ray system.

FIG. 2 depicts a representation of the acts of FIG. 1 in examples.

FIG. 3 to FIG. 6 depict examples of views of objects for insertion into the body of a patient with encoded markings.

FIG. 7 depicts an example of a medical system for carrying out the method described herein.

### DETAILED DESCRIPTION

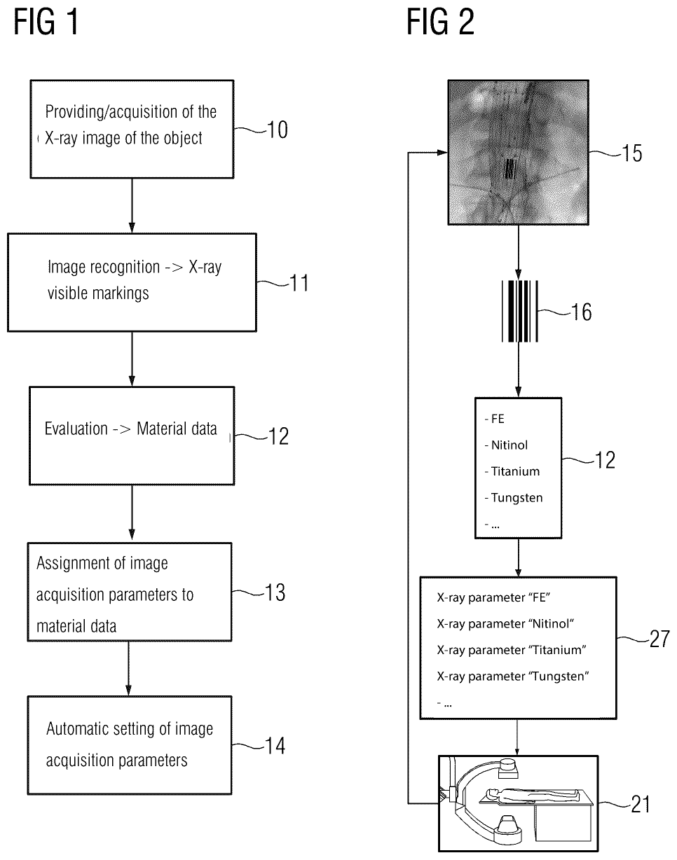

FIG. 1 shows acts of the method for automatically setting image acquisition parameters of a medical X-ray system when acquiring X-ray images of a patient with an object arranged on the body or in the body of the patient.

During interventional procedures on a patient in which a medical object is placed on or in the body of the patient (for example, in a vascular system, bone or tissue), the procedure may be monitored and observed by X-ray imaging, for example, fluoroscopy, by an X-ray machine. The method is particularly suitable for such applications because the method minimizes the X-ray dose while optimizing the detectability of the object and providing acceptable image quality.

A prerequisite for the method is the application of encoded information relating to the material composition of the object on the object. The encoded markings are X-ray visible. One-dimensional or two-dimensional bar codes or other binary codes that are easy to read optically are particularly advantageous here. Other machine-readable codes may also be used. These may be easily placed on medical objects such as guide wires, catheters, vascular prostheses, stents, stent grafts, shunts, mitral clips, heart valves, prostheses, nails, or screws. The attachment may be carried out at the factory or by the hospital or user in order to prepare usable medical objects for an X-ray-supervised procedure. In particular, the encoding contains information relating to the materials and compounds that make up the object and/or their exact composition. The markings may be applied, printed, adhered, notched, punched, welded, etc.

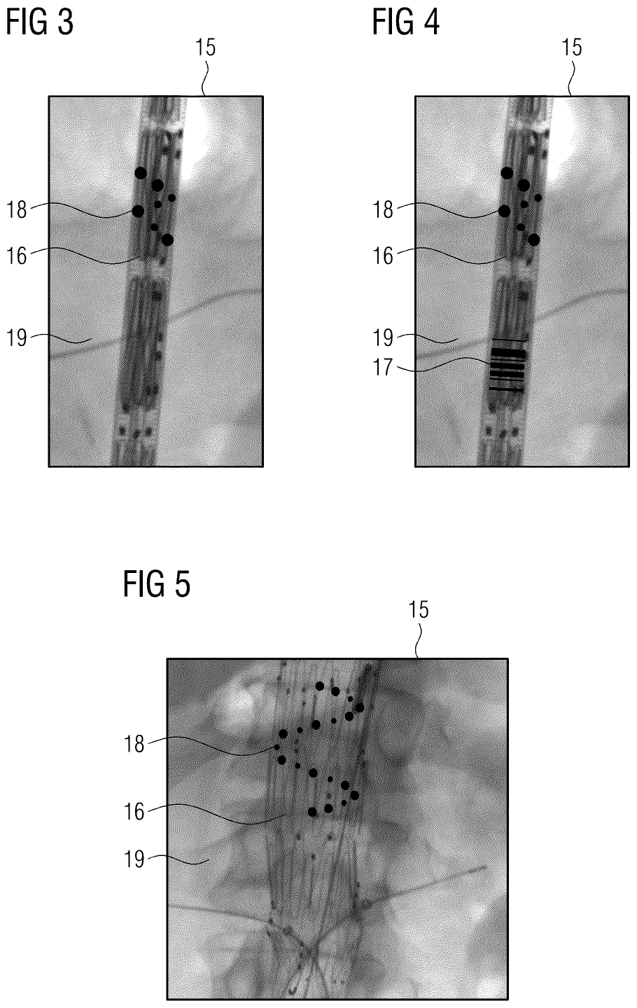

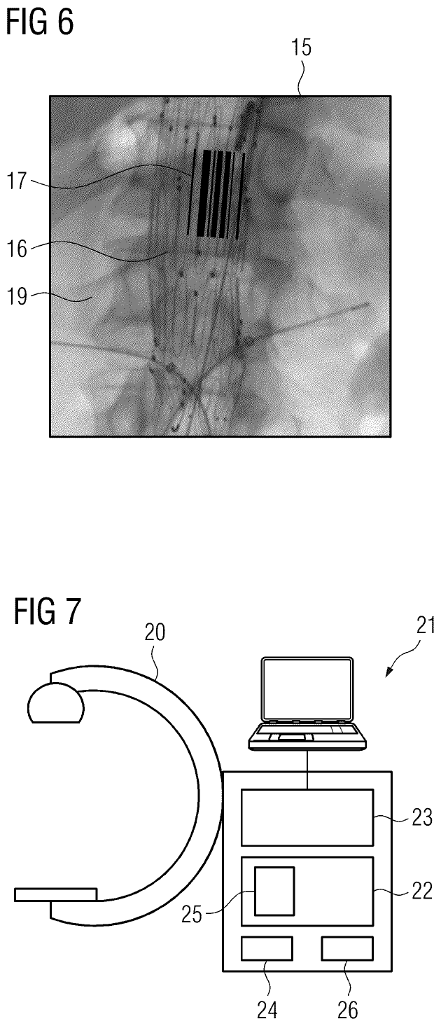

In act 10 of the method, at least one X-ray image of the object is provided, wherein the object has X-ray visible markings on its surface with encoded information relating to material data of the object. The X-ray image may be provided from a memory, for example, or an X-ray image may be acquired and used live by the X-ray machine performing the monitoring. The stored X-ray image may have been previously acquired and stored, for example, as a pre-operative process, by the same X-ray machine or also by a different X-ray machine. The X-ray image may be a two-dimensional projection X-ray image (for example, fluoroscopy or radiography) or a 3D volume X-ray image. Examples of X-ray images 15 are shown in FIGS. 3 to 6, in each of which stents 16 are shown that are inserted into a vascular system of a patient 19. FIGS. 4 and 6 each show one-dimensional bar codes 17 on the stents 16. FIG. 4 also shows a binary code 18. FIGS. 3 and 5 only show binary codes 18. The binary codes shown are merely examples. They include spheres in two different sizes, one of which represents a “0” and the other a “1,” and are also arranged in a circular or spiral shape. This results in a wave pattern that is also visible in a projection from all directions and may be clearly read in terms of the sequence.

In act 11, the X-ray image is image-processed, for example, via an image system 22 using software in order to recognize the X-ray visible markings of the object. For example, an image recognition algorithm which is stored in the image system is used for this purpose. The image processing is carried out automatically based on the acquired X-ray image of the object. In addition further image processing may also be carried out, for example, segmenting the object.

In act 12, the X-ray visible markings recognized with the aid of the X-ray image are evaluated with respect to the information contained therein and relating to material data of the object. Thus, the bar code or binary code, for example, is decoded by an evaluation unit 25 and the appropriate material information identified. The evaluation unit 25 may also use an algorithm for this purpose. FIG. 2 shows an example in which it is possible with the aid of the bar code 16 to evaluate that the object is made of 100% Nitinol. Act 11 and act 12 may also be carried out jointly by an image recognition algorithm. Image recognition algorithms are known for this purpose.

In act 13, the identified material data is then automatically assigned optimized image acquisition parameters for the X-ray machine carrying out the monitoring. This may be done, for example, via a system-internal or external list or internal or external database in which optimized image acquisition parameters are stored for a variety of possible material data. The lists or data may have previously been created experimentally or empirically, created from simulations, or by machine learning and compiled. System-internal or publicly accessible data or lists may be used. FIG. 2 shows an excerpt 27 from such a list as an example: specific image acquisition parameters are assigned to the material “Nitinol.” The image acquisition parameters may include X-ray current, X-ray voltage, X-ray dose, exposure time, filter setting (e.g., thickness, position, shape of the filters, etc.), collimator setting, X-ray pulse length.

The list/database may contain in addition further selection options, for example, the type of imaging (fluoroscopy, radiography, 3D imaging, etc., photon-counting or integrating X-ray detector, etc.) and/or patient characteristics (size, weight, etc., for example, via a patient model). Accordingly, further information may also be used for a selection in this regard, for example, queried by a user or obtained from another source of information (e.g., system control, sensor data, etc.). The list/database may be as complex as desired or only include a few entries.

Once the assigned image capture parameters have been queried, the parameters are then set automatically in act 14. For this purpose, the previously set image acquisition parameters are changed if they do not match. In a further act, another X-ray image may then be taken with the newly set image acquisition parameters. The image acquisition parameters are optimized for capturing the object so that it is optimally imaged in the further X-ray image. This creates a closed control loop.

An iterative method is also conceivable, in which the further X-ray image is also analyzed with regard to encoded markings of the same object or a further object, material data is decoded, and X-ray imaging parameters are assigned and set. This may be particularly advantageous if the encoded markings are difficult to recognize on the previous X-ray image or if multiple objects are present. The method may be repeated iteratively until a cancellation criterion is achieved. Such a cancellation criterion may be a user input or the end of a fluoroscopy sequence or the procedure.

In addition, the ability of the medical object to be recognized in the initial X-ray image may also be compared with that in the further X-ray image (with the assigned X-ray imaging parameters) and a corresponding measure may then be taken (for example, resetting one or more X-ray imaging parameters).

An encoding of the material data may be “public knowledge,” i.e. publicly accessible, or also individually accessible only to the users of a specific medical X-ray system.

FIG. 7 shows the medical X-ray system 21 for carrying out the method. The X-ray system 21 has an X-ray machine, for example, with an imaging system in the form of a C-arm 20 with an X-ray detector and an X-ray source for acquiring X-ray images. The X-ray system 21 is actuated by a system control unit 23, for example, X-ray imaging parameters are set by the system control unit 23. Furthermore, the X-ray system 21 has an image system 22 for image processing X-ray images, in particular using an algorithm/software for image recognition. In addition, the X-ray system 21 has an evaluation unit 25 for evaluating the X-ray visible markings with respect to information relating to material data of the object, the evaluation unit 25 may also be part of the image system 22. In this case, a further algorithm/further software may be provided or the algorithm/software for image recognition may carry out the evaluation simultaneously. Furthermore, the X-ray system has an assignment unit 26 on which image acquisition parameters, which are stored for example in a list in a memory unit 24, are assigned to the material data.

The X-ray system may also have a robotic navigation system for navigating medical objects through the body of a patient in conjunction with an interventional procedure.

The disclosure may be briefly summarized in the following manner. For optimizing an X-ray dose and image quality, a method is provided for automatically setting image acquisition parameters of a medical X-ray system when acquiring X-ray images of a patient with an object arranged on or in the body of the patient. The method includes: a) providing an X-ray image of the object, wherein the object has X-ray visible markings on its surface with encoded information, (e.g., material data), relating to the object; b) using image recognition for recognizing the X-ray visible markings of the image of the object; c) evaluating the X-ray visible markings with regard to the information contained therein relating to the object; d) assigning image acquisition parameters to the information; and e) automatically setting of the assigned image acquisition parameters on the X-ray system. The method automates the selection of the material-dependent fluoroscopy parameters and guarantees optimization to the medical object actually visible in the X-ray image.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for automatically setting image acquisition parameters of a medical X-ray system when acquiring X-ray images of a patient with at least one object arranged on or in a body of the patient, the method comprising:
providing an X-ray image of the object, wherein the object has X-ray visible markings on a surface of the object with encoded information relating to the object;
determining the X-ray visible markings of the X-ray image of the object;
evaluating the X-ray visible markings with regard to the encoded information related to the object;
assigning image acquisition parameters to the encoded information; and
automatically setting the assigned image acquisition parameters on the medical X-ray system.

2. The method of claim 1, wherein the encoded information comprises material data relating to the object.

3. The method of claim 1, wherein the determining of the X-ray visible markings is performed by image recognition.

4. The method of claim 1, further comprising:
acquiring at least one further X-ray image using the set image acquisition parameters.

5. The method of claim 4, further comprising:
repeating the determining, the evaluating, the assigning, and the automatically setting using the at least one further X-ray image.

6. The method of claim 5, wherein the method is repeated iteratively until a cancellation criterion is achieved.

7. The method of claim 1, wherein the encoded information is encoded as a one-dimensional bar code, a multi-dimensional bar code, a binary code, or a machine-readable code.

8. The method of claim 1, wherein the assigning is carried out based on a comparison with a stored list or data from a database.

9. The method of claim 1, wherein the image acquisition parameters comprise an X-ray current, an X-ray voltage, an X-ray dose, an exposure time, a filter setting, a collimator setting, an X-ray pulse length, or a combination thereof.

10. The method of claim 1, wherein additional patient information is taken into consideration when automatically setting the assigned image acquisition parameters.

11. The method of claim 1, wherein in addition an adaptation to a type of acquisition is taken into consideration when setting the assigned image acquisition parameters.

12. An object for insertion into a body of a patient, the object comprising:
at least one X-ray visible marking comprising encoded information relating to the object.

13. The object of claim 12, wherein the encoded information comprises material data relating to the object.

14. A medical X-ray system comprising:
an X-ray machine configured to acquire X-ray images of an object, wherein the object has X-ray visible markings on a surface of the object with encoded information relating to the object;
an image system configured to carry out an image recognition using an algorithm and determine the X-ray visible markings of the X-ray image of the object;
an evaluation unit configured to evaluate the X-ray visible markings with regard to the encoded information relating to the object;
an assignment unit configured to assign image acquisition parameters to the encoded information;
a system control unit configured to set the assigned image acquisition parameters for the X-ray machine; and
a memory with a stored list or a data link to a database, wherein the memory is configured to store the assigned image acquisition parameters for the X-ray machine.

15. The medical X-ray system of claim 14, wherein the encoded information comprises material data relating to the object.
